(19) United States
(12) Patent Application Publication
(10) Pub. No.: US 2024/0287440 A1
(43) Pub. Date: Aug. 29, 2024

(54) MICROALGAE-BASED PRODUCTS WITH IMPROVED NUTRIENT BIOACCESSIBILITY AND OXIDATIVE STABILITY

(71) Applicant: SOCIETE DES PRODUITS NESTLE S.A., Vevey (CH)

(72) Inventors: GRETA CANELLI, Lausanne (CH); FABIOLA DIONISI, Epalinges (CH); CHRISTOPH JOSEF BOLTEN, Rielasingen (DE); PATRICIA MURCIANO MARTINEZ, Pully (CH); ALEXANDER MATHYS, Zurich (CH); LEANDRO BUCHMANN, Seuzach (CH); LUC JAQUENOD, Zurich (CH); ISABELLE KUSTER, Zurich (CH)

(21) Appl. No.: 18/292,073

(22) PCT Filed: Jul. 27, 2022
(86) PCT No.: PCT/EP2022/071103

§ 371 Date: Jan. 25, 2024

(30) Foreign Application Priority Data
Jul. 28, 2021 (EP) 21188331.9

(51) Int. Cl. C12N 1/12; A23L 2/00; A23L 29/00; C12N 1/06; C12N 13/00

(52) U.S. Cl. CPC C12N 1/12; A23L 2/00; A23L 29/065; C12N 1/06; C12N 13/00

(57) ABSTRACT

The invention relates to a method of making a product for human consumption which comprises partially lysed microalgae, said method comprising a) preparing a suspension of microalgae, wherein the microalgae belong to a phylaselected from Chlorophyta, Ochrophyta, and Heterokonta; b) applying a pulsed electric field to the suspension of microalgae, wherein said pulsed electric field has a specific energy input between 25 to 150 kJ per kg suspension (kg sus⁻¹); c) optionally treating the microalgae with enzyme; d) incubating the biomass suspension at 300 rpm, 4-37° C. for 6-48 h e) forming a biomass of partially lysed microalgae; and f) adding the biomass of partially lysed microalgae to a product for human consumption.

### INTRODUCTION

Microalgae are getting increasing attention as an alternative omega3-PUFAs source, being primary producers of these high value compounds. Nowadays microalgae omega3-PUFAs rich lipids are mainly used upon extraction. On the other hand, dried powders of microalgae biomass (rich in protein and omega3-PUFAs), mainly Chlorella vulgaris and Arthospira platensis, commonly known as Spirulina, can already be found on the market. However, use of whole cells leads to a limited lipid bioaccessibility. In addition, omega3-PUFAs are very susceptible to oxidation, due to their large number of double bonds. Oxidation leads to off-flavors formation and loss of nutritional value.

Maintaining the integrity of the whole cell structure in microalgae has been shown to protect omega3-PUFAs from oxidation during wet storage post-harvest. However, the presence of an intact cell wall may limit omega3-PUFAs bioaccessibility, because of the indigestible polysaccharides.

There is a clear need to enhance nutrient bioaccessibility and oxidative stability in microalgae-based products.

### Embodiments of the Invention

The presented invention improves the prior art and addresses the abovementioned need by using pulsed field and enzymatic treatment of the cell wall.

The invention relates in general to a method of making a product for human consumption which comprises lysed microalgae, in particular partially lysed microalgae.

More specifically, the invention relates to a method of making a product for human consumption which comprises partially lysed microalgae, said method comprising applying a pulsed electric field to a suspension of microalgae and forming a biomass of partially lysed microalgae.

More specifically, the invention relates to a method of making a product for human consumption which comprises partially lysed microalgae, said method comprising a) preparing a suspension of microalgae; b) applying a pulsed electric field to the suspension of microalgae; and c) forming a biomass of partially lysed microalgae.

More specifically, the invention relates to a method of making a product for human consumption which comprises partially lysed microalgae, said method comprising a) preparing a suspension of microalgae; b) applying a pulsed electric field to the suspension of microalgae; c) forming a biomass of partially lysed microalgae; and d) adding the biomass of partially lysed microalgae as an ingredient of a product for human consumption.

More specifically, the invention relates to a method of making a product for human consumption which comprises partially lysed microalgae, said method comprising a) preparing a suspension of microalgae, wherein the microalgae belong to a phyla selected from Chlorophyta, Ochrophyta, and Heterokonta; b) applying a pulsed electric field to the suspension of microalgae; c) forming a biomass of partially lysed microalgae; and d) adding the biomass of partially lysed microalgae as an ingredient of a product for human consumption.

More specifically, the invention relates to a method of making a product for human consumption which comprises partially lysed microalgae, said method comprising a) preparing a suspension of microalgae, wherein the microalgae belong to a phyla selected from Chlorophyta, Ochrophyta, and Heterokonta; b) applying a pulsed electric field to the suspension of microalgae, wherein said pulsed electric field has a specific energy input between 25 to 150 kJ per kg suspension (kgₛᵤₛ⁻¹); c) forming a biomass of partially lysed microalgae; and d) adding the biomass of partially lysed microalgae as an ingredient of a product for human consumption.

In one embodiment, the invention relates to a method of making a product for human consumption which comprises partially lysed microalgae, said method comprising a) preparing a suspension of microalgae, wherein the microalgae belong to a phyla selected from Chlorophyta, Ochrophyta, and Heterokonta; b) applying a pulsed electric field to the suspension of microalgae, wherein said pulsed electric field has a specific energy input between 25 to 150 kJ per kg suspension (kg ₛᵤₛ⁻¹); c) optionally adding enzyme to the suspension of microalgae; d) forming a biomass of partially lysed microalgae; and e) adding the biomass of partially lysed microalgae as an ingredient of a product for human consumption.

In one embodiment, the phyla is Chlorophyta. In one embodiment, the phyla is Ochrophyta. In one embodiment, the phyla is Heterokonta.

In an embodiment, the microalgae belong to the species Chlorella. In an embodiment, the microalgae belong to the species Auxenochlorella.

In an embodiment, the microalgae is Chlorella vulgaris, preferably CCALA 256.

In an embodiment, the enzyme is a galactanase, for example endo-1,4-β-galactanase.

In an embodiment, the enzyme is a rhamnohydrolase, for example rhamnogalacturonan rhamnohydrolase.

In an embodiment, the enzyme is a chitinase.

In an embodiment, the enzyme is endo-1,4-β-galactanase and rhamnogalacturonan rhamnohydrolase.

In an embodiment, the enzyme is endo-1,4-β-galactanase, rhamnogalacturonan rhamnohydrolase and/or chitinase.

In an embodiment, the temperature of the suspension of microalgae before applying the pulsed electric field is between 2 to 30° C.

In an embodiment, the pulsed electric field has a specific energy input between 25 to 100 kJ kgₛᵤₛ⁻¹.

In an embodiment, the pulsed electric field has a specific energy input of between 30 to 35 kJ kg SUS-1, for example about 32 kJ kgₛᵤₛ⁻¹.

In an embodiment, the pulsed electric field has an electric field strength between 10 kV cm⁻¹ to 45 kV cm⁻¹, preferably between 10 kV cm⁻¹ to 35 kV cm⁻¹, more preferably between 20 kV cm⁻¹ to 30 kV cm⁻¹.

In an embodiment, the pulsed electric field has an electric field strength of between 20 to 25 kV cm⁻¹.

In an embodiment, the pulsed electric field has a pulse length between 5 μs to 25 μs.

In an embodiment, the pulsed electric field has an electric field strength of between 20 to 25 kV cm⁻¹ and pulse length of 5 μs.

In an embodiment, the pulsed electric field has a pulse number between 5 and 30 applied, preferably a pulse number of 10 is applied.

In an embodiment, the pulsed electric field comprises bipolar square wave electric pulses. In an embodiment, the pulsed electric field comprises unipolar electric pulses. In an embodiment, the pulsed electric field comprises exponential decay electric pulses. Preferably, the pulsed electric field comprises bipolar square wave electric pulses.

In one embodiment, the microalgae are incubated after PEF treatment, for example at about 4° C., or at about 25° C., or at about 37° C.

The typical incubation time may be for up to about 72 hours, for example between 1 to 72 hours, or between 6 to 72 hours, or between 12 to 72 hours, or between 18 to 72 hours, or between 24 to 72 hours, or between 48 to 72 hours. The typical incubation time may be about 1, about 6, about 12, about 18, about 24, about 48, or about 72 hours.

The suspension may be incubated with agitation, for example at about 300 rpm.

In an embodiment, the microalgae are incubated after PEF treatment at about 4° C. for at least 24 hours, for example for about 48 hours.

In an embodiment, the microalgae are incubated after PEF treatment at about 25° C. or at about 37° C. for at least 6 hours, for example for about 12 hours.

In an embodiment, the microalgae are collected, preferably by centrifugation, and re-suspended in buffer after applying the pulsed electric field, wherein the buffer has a temperature between 4° C. to 37° C., and the microalgae are incubated in the buffer between 6 to 72 hours, preferably 12 to 72 hours.

In an embodiment, the buffer is a phosphate buffer, preferably a 0.05 M potassium phosphate buffer at pH 6.

In an embodiment, the microalgae have a mean particle size between 3 μm to 6 μm after applying the pulsed electric field or enzyme treatment.

In an embodiment, the microalgae have a mean particle size between 3 μm to 6 μm after applying the pulsed electric field and enzyme treatment.

The invention further relates to a method for improving lipid bioaccessibility of microalgae for human consumption, said method comprising a) preparing a suspension of microalgae, wherein the microalgae belong to a phyla selected from Chlorophyta, Ochrophyta, and Heterokonta; b) applying a pulsed electric field to the suspension of microalgae, wherein said pulsed electric field has a specific energy input between 25 to 150 kJ per kg suspension (kg ₛᵤₛ⁻¹); and c) optionally adding enzyme to the suspension of microalgae; characterized in that the suspension of microalgae is incubated at between 4° C. to 37° C. for between 6 h to 48 h after step b) and/or step c).

In one embodiment, the suspension of microalgae is incubated with agitation, for example at 300 rpm.

The invention further relates to a method for preserving lipid oxidative stability in microalgae for human consumption, said method comprising a) preparing a suspension of microalgae, wherein the microalgae belong to a phyla selected from Chlorophyta, Ochrophyta, and Heterokonta; b) applying a pulsed electric field to the suspension of microalgae, wherein said pulsed electric field has a specific energy input between 25 to 150 kJ per kg suspension (kgₛᵤₛ⁻¹); c) optionally adding enzyme to the suspension of microalgae.

The invention further relates to a product for human consumption comprising a biomass of microalgae, said product made by a method according to the invention.

In one embodiment, the product is for human consumption, for example as an RTD beverage.

### DETAILED DESCRIPTION

### Microalgae Preparation

Preferably, microalgae is cultivated in bold basal medium. The medium can be supplemented with about 20 g L-1 glucose. The microalgae may be grown in the dark, for example at about 25° C. Preferably, the microalgae is shaken whilst growing, for example at about 150 rpm in a shaking incubator. The microalgae may be harvested, for example upon reaching stationary phase, typically after about four days of cultivation. The microalgae is then typically centrifuged, for example at about 10000 g, for about 10 min, at about 4° C. The resulting pellet is used for pulsed electric field treatment. Preferably, the microalgae is C. vulgaris, for example CCALA 256. This microalgae may be purchased from the Culture Collection of Autotrophic Organisms (Třeboň, Czech Republic).

### Pulsed Electric Field

Typically, microalgae concentration is about 70 g L⁻¹ prior to PEF treatment. The microalgae pellet is typically resuspended in potassium phosphate buffer, for example at about pH 6. Conductivity (6) may be adjusted to about 2 mS cm-1. PEF treatment of microalgae suspensions may use plate-plate electroporation cuvettes. The electrode distance may be about 4 mm. The applied voltage may vary between 8 kV and 12 kV. The resulting electric field strengths may be between 20 kV cm-1 and 30 kV cm-1. Pulse widths of 5 μs up to 25 μs may be applied. A pulse number of 10 may be applied. Following PEF treatment, the microalgae can be incubated at different temperatures (for example about 4, 25, or 37° C.) for different times (0, 1, 6, 12, 18, 24, 48, 72 h) at about 300 rpm. The microalgae biomass may then be snap-frozen with liquid nitrogen.

### Adding Enzyme/Enzymatic Treatment

Prior to enzymatic treatment, microalgae are typically suspended in potassium phosphate buffer. The buffer concentration is typically about 50 mM. The buffer typically is about pH 6. The microalgae concentration may be about 20 g L-1. Chitinase, rhamnogalacturonan rhamnohydrolase, and/or endo-1,4-β-galactanase may be added to the microalgae suspension. The endo-1,4-β-galactanase may be in a potassium phosphate buffer, at about pH 6. The suspensions may then be incubated at 37° C. at about 300 rpm for about 24 h. After the incubation, the biomass may be snap-frozen with liquid nitrogen.

PEF Combined with Enzymatic Treatment

Microalgae biomass are typically treated with a pulse width of about 5 μs. The electric field strength may be about 20 kV cm-1. 10 pulses may be used. Enzymes may be added before diluting the suspension to about 20 g L⁻¹ with potassium phosphate buffer, for example 50 mM potassium phosphate buffer at pH 6. Chitinase, rhamnogalacturonan rhamnohydrolase, and/or endo-1,4-β-galactanase may be added. After mixing, the samples are typically incubated at 37° C. for about 24 h at about 300 rpm. After the incubation, the biomass may be immediately snap-frozen.

### Definitions

When a composition is described herein in terms of wt %, this means a mixture of the ingredients on a moisture free basis, unless indicated otherwise.

As used herein, the term “about” is understood to refer to numbers in a range of numerals, for example the range of −30% to +30% of the referenced number, or −20% to +20% of the referenced number, or −10% to +10% of the referenced number, or −5% to +5% of the referenced number, or −1% to +1% of the referenced number. All numerical ranges herein should be understood to include all integers, whole or fractions, within the range.

Those skilled in the art will understand that they can freely combine all features of the present invention disclosed herein. In particular, features described for the compositions of the present invention may be combined with the method or uses of the present invention and vice versa. Further, features described for different embodiments of the present invention may be combined. Where known equivalents exist to specific features, such equivalents are incorporated as if specifically referred to in this specification.

Further advantages and features of the present invention are apparent from the figures and non-limiting examples.

### EXAMPLES

The following examples are illustrative of some of the products and methods of making the same falling within the scope of the present invention. Changes and modifications can be made with respect to the invention. The skilled person will recognize many variations in these examples to cover a wide area of formulas, ingredients, processing and mixtures to rationally adjust the nutrients and other elements of the invention for a variety of applications.

### Example 1

### Microalgae Cultivation

C. vulgaris (CCALA 256) was purchased from the Culture Collection of Autotrophic Organisms (Třeboň, Czech Republic). C. vulgaris was cultivated in biological triplicate (duplicate for the lipid extractability experiment) in bold basal medium supplemented with 20 g L⁻¹ glucose, in 500 mL Erlenmeyer flasks (working volume 250 mL) in the dark, at 25° C. and 150 rpm in a shaking incubator (Multitron Pro, Infors AG, Bottmingen, Switzerland). The biomass was harvested after four days of cultivation when reaching the stationary phase and centrifuged (10000 g, 10 min, 4° C.). The supernatant was discarded and the pellet (fresh biomass) was used for further experiments.

The growth of C. vulgaris was monitored by optical density (OD) measurements at 750 nm (GENESYS™ 10S, Thermo Fischer Scientific Inc., Waltham, MA, USA). The correlation factor between OD and dry weight (DW) is shown by the equation (R²=0.9796).

$$D W [ g {L}^{- 1} ] = 0.5596 \cdot {OD}_{750 n m} + 0.4179$$

### Example 2

### Pulsed Electric Field

Prior to cell electroporation, the concentration and conductivity of the microalgae cultures were standardized. For all PEF experiments, microalgae concentration was set at 70 g L⁻¹.

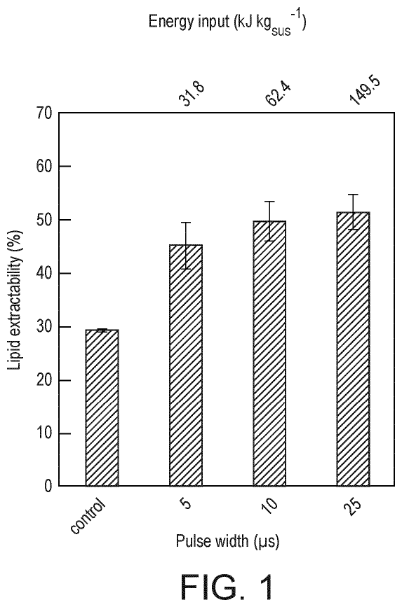

The fresh biomass pellet was resuspended in potassium phosphate buffer at pH 6, adjusted to a conductivity (6) of 2 mS cm⁻¹. The conductivity adjustment ensured matched load conditions in subsequent PEF treatments. Directly after the preparation of the microalgae suspensions, the conductivity was measured, which resulted in 1.7 mS cm⁻¹. After vortexing, 1 mL aliquots of the microalgae suspensions were transferred to plate-plate electroporation cuvettes (VWR International bvba., Leuven, Belgium) for PEF treatment. The samples were treated batch wise at 4 mm electrode distance. The experimental setup consisted of a cuvette holder connected to a RUP6-15CL pulse generator (GBS-Elektronik, Radeberg, Germany). Pulse measurements were conducted with a P6015A voltage probe (Tektronix Inc., Beaverton OR, USA) connected to a Wave Surfer 10 oscilloscope (Teledyne LeCroy GmbH, Heidelberg, Germany). The applied voltage was varied between 8 kV and 12 kV resulting in electric field strengths of 20 kV cm⁻¹ and 30 kV cm⁻¹, respectively. Pulse widths of 5 μs up to 25 μs and a pulse number of 10 were applied. Different combinations were tested systematically to investigate their impact on lipid extractability. Following the PEF treatment, the biomass coming from several cuvettes was combined and incubated at different temperatures (4, 25, 37° C.) for different times (0, 1, 6, 12, 18, 24, 48, 72 h) at 300 rpm. After the incubation, the biomass was immediately snap-frozen with liquid nitrogen and stored at −20° C. until further analysis. Untreated samples (control) were concentrated and standardized to the same conductivity, incubated and then snap-frozen. FIG. 1 shows the lipid extractability (%) of C. vulgaris biomasses treated at 20 kV cm⁻¹ and different pulse widths (5-25 μs), and biomasses treated at 5 μs and different electric field strengths (20-30 kV cm⁻¹).

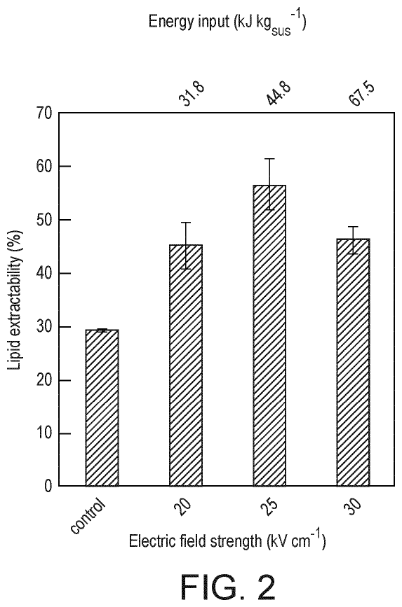

The effect of the field strength was assessed (20-30 kV cm⁻¹) at a constant pulse width of 5 μs (FIG. 2). In both experiments, biomass suspension was incubated for 1 h at 25° C. after PEF treatment. The lipid extractability was enhanced by PEF treatment at 5 μs compared to the untreated control. Treatments at higher pulse widths, and therefore higher energy inputs, did not cause an improvement of the lipid extractability. The lipid extractability was assessed at various electric field strengths and at a constant pulse width of 5 μs. When the biomass was treated at 20 kV cm⁻¹, the lipid extractability was 15.8% higher than the control, but the highest increase was reached when treating at 25 kV cm⁻¹ (+26.9%). At higher electric field strengths (30 kV cm⁻¹) and therefore greater energy inputs, the lipid extractability was reduced. Noteworthy, when treating at 25 kV cm⁻¹ or higher, system instabilities occurred. Pulses of 5 μs at 20 kV cm⁻¹ represent a μsPEF treatment with an energy input of 31.8 kJ kg ₛᵤₛ⁻¹.

### Example 3

### Enzymatic Treatment

Fresh microalgae biomass was suspended in 50 mM potassium phosphate buffer at pH 6, at 20 g L⁻¹. Solution of chitinase (700 μL, 1 mg mL⁻¹ in microalgae suspension, Sigma-Aldrich, Switzerland), rhamnogalacturonan rhamnohydrolase (35 μL, NZYTech, Portugal), endo-1,4-β-galactanase (1.75 μL, 750 U mL⁻¹ in potassium phosphate buffer, pH 6, Megazyme, Ireland) were added to the microalgae suspension. After mixing, the samples were incubated at 37° C. at 300 rpm for 24 h on a stirring plate. After the incubation, the biomass was immediately snap-frozen with liquid nitrogen and stored at −20° C. until further analysis. For the control (untreated biomass), the enzyme solution was replaced by potassium phosphate buffer (pH 6, 50 mM). All tubes were incubated for 24 h at 37° C. on a stirring plate at 300 rpm. After incubation, the samples were collected and immediately cooled in crushed ice.

### Example 4

PEF Combined with Enzymatic Treatment

The microalgae biomass was treated with a pulse width of 5 μs, electric field strengths of 20 kV cm⁻¹, 10 pulses. The biomass coming from several electroporation cuvettes was combined and the following enzymes were added before diluting the suspension to 20 g L⁻¹ with potassium phosphate buffer (50 mM, pH 6): Chitinase (700 μL, 1 mg mL⁻¹ in potassium phosphate buffer, pH 6), rhamnogalacturonan rhamnohydrolase (35 μL, NZYTech, Portugal), endo-1,4-β-galactanase (1.75 μL, 750 U mL⁻¹ in potassium phosphate buffer, pH 6, Megazyme, Ireland). After mixing, the samples were incubated at 37° C. for 24 h at 300 rpm on a stirring plate. After the incubation, the biomass was immediately snap-frozen with liquid nitrogen and stored at −20° C. until further analysis.

### Example 5

### Lipid Extractability

The lipid extractability was assessed by measuring the hexane: isopropanol (HI) extraction efficiency. HI extraction efficiency is expressed as the ratio of the free lipids extracted by hexane:isopropanol (3:2 v v⁻¹) compared to the total lipids determined by direct transesterification, expressed as a percentage. HI does not easily penetrate intact rigid microalgal cells, resulting in a lower extraction yield.

Hexane:isopropanol (3:2 v v⁻¹, 1.5 mL) was added to 10 mg of freeze-dried PEF-treated microalgal biomass and the mixtures were vortexed for 30 s. The samples were centrifuged (10 min, 750 g, 25° C.) and the solvent layer containing the extracted lipids was transferred to a weighed flask. In total, these extraction steps were performed four times. All solvent layers were combined, and subsequently the solvent was removed by evaporation under a nitrogen stream. The extracted lipids as well as the total lipids in the dried biomass were quantified by gas chromatography (GC) upon transesterification to fatty acids methyl esthers (FAMEs). In brief, fatty acids were directly trans-esterified using 1.5 N methanolic hydrochloric acid solution and analysed by gas chromatography using an instrument equipped with a split-injection port and flame ionization detection (FID) (7890A; Agilent Technologies, Basel, Switzerland). The following temperature-time program was used: 50° C. (0.2 min), 50-180° C. (120° C. min⁻¹), 180-220° C. (6.7° C. min⁻¹), and 220-250° C. (30° C. min⁻¹) on a 70% cyanopropyl polysilphenylene-siloxane column with a length of 10 m, internal diameter of 0.1 mm, and film of 0.2 μm (BPX70; SGE Analytical Science, Milton Keynes, UK). Peak identification was performed by comparing the retention times with FAME standards (Nu-Chek Prep. Inc., Elysian, USA). The peak areas were quantified with OpenLab CDS VL software (Agilent Technologies, Basel, Switzerland).

### Example 6

### Lipid Bioaccessibility

Lipid bioaccessibility was measured by an in vitro digestion model, following the standardized protocol (INFOGEST 2.0). In brief, digestion was performed in triplicate (n=3) at 37° C. with stirring at 300 rpm. To simulate the oral phase (2 min, pH 7), the following solutions were mixed with the freeze-dried biomass (0.3 g): water (1.13 mL), simulated salivary fluid (SSF, 0.96 mL), and CaCl₂ (6 μL, 0.3 M). To initiate the gastric phase, simulated gastric fluid (SGF, 1.92 mL) and CaCl₂ (1.2 μL, 0.3 M) were added to the oral bolus, and the pH was set to 3. Pepsin (0.12 mL, 80000 U mL⁻¹; Sigma-Aldrich, Buchs, Switzerland) and gastric lipase (0.12 mL, 2400 U mL⁻¹; Lipolytech, Marseille, France) were added, and the final volume was topped up to 4.8 mL with water. The pH was regularly adjusted to 3. After 2 h of incubation, the pH was set to 7, and simulated intestinal fluid (SIF, 2.04 mL), CaCl₂ (9.6 μL, 0.3 M), pancreatin (1.2 mL, 800 U mL⁻¹; Sigma-Aldrich), and bile salts (0.6 mL, 0.16 mM; Sigma-Aldrich) were added. Water was added to a final volume of 9.6 mL. After 2 h of incubation, an aliquot (2 mL) of full digesta was frozen with liquid nitrogen and freeze-dried. The remaining full digesta was centrifuged (30 min, 10000 g, 4° C.). The micellar phase (supernatant) and the pellet were separately frozen with liquid nitrogen and freeze-dried. Infant formula (Aptamil 1; Milupa, Dublin, Ireland) was used as positive control and subjected to in vitro digestion, as entire bioaccessibility was anticipated for this sample. Water (1.2 mL) without microalgal biomass was digested as a blank to quantify the fatty acids coming from the digestive fluids and enzymes.

The full digesta and micellar phase were analysed in terms of lipid content. The lipid content was expressed as the total fatty acids measured. Lipid bioaccessibility was defined as the amount of lipid contained into the micellar phase (corrected by the lipid in the micellar phase of the enzyme blank) divided by the amount of lipid in the full digesta (corrected by the lipid in the full digesta of the enzyme blank, respectively), expressed as a percentage (%).

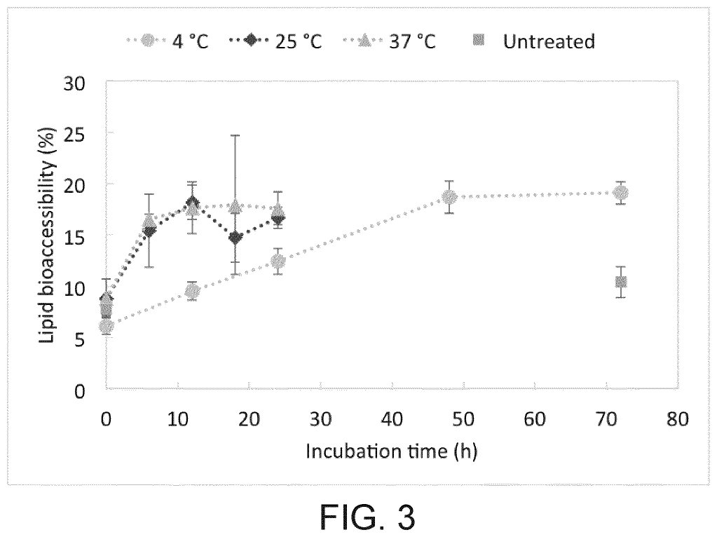

Lipid bioaccessibility by an in vitro digestion model of C. vulgaris biomass that was PEF treated (5 μs at 20 kV cm-1) was anaysed, followed by incubation conditions at different temperature (4, 25, 37° C.) and for up to 72 h (FIG. 3). After the incubation, the algae suspension was immediately snap frozen in liquid nitrogen and freeze-dried for the following in vitro digestion. This combination of parameters was selected among the feasible ones previously tested in this study, as yielding to the highest lipid extractability.

FIG. 3 shows lipid bioaccessibility (%) of C. vulgaris biomass after PEF treatment (5 μs at 20 kV cm⁻¹), followed by an incubation of at 4° C. (●), 25° C. ( ), and 37° C. (▴). The lipid bioaccessibility of untreated biomass without and with incubation (4° C., 72 h) is also shown (▪). Error bars show the standard deviation between biological triplicates (n=3).

Incubation (4° C., 72 h) without PEF treatment led to a lipid bioaccessibility of 10.4±1.7%, which is lower than values obtained for PEF treated samples followed by incubation of at least 6 h at 25° C. or 37° C. and incubation of at least 24 h at 4° C. Longer incubation time after PEF treatment caused an increase in lipid bioaccessibility, for any incubation temperature. FIG. 3 shows a plateau of maximum lipid bioaccessibility at 18-19%. This value was reached after 12 h of incubation for the biomass kept at either 25° C. or 37° C. Longer incubation time (48 h) was necessary at 4° C. to yield a lipid bioaccessibility of 18.7±1.6%. Without wishing to be bound by theory, the reason why no further increase could be reached could be that the aqueous incubation of the PEF-treated algae led to the fusion of the lipid droplets in a big unique body which was harder to access by the digestive enzymes.

An increase in lipid bioaccessibility with longer incubation times would agree with the theory of PEF triggering the release of autolytic enzymes that are subsequently degrading the cell wall, favouring an increase in the bioaccessibility of lipids. Moreover, the kinetic behaviour of the lipid bioaccessibility was proportional to the temperature, which further suggests the role played by endogenous enzymes in such a mechanism, being enzymatic activity temperature dependent. Such an enzymatic-driven process may indeed be slowed down at non-physiological temperatures, i.e., 4° C.

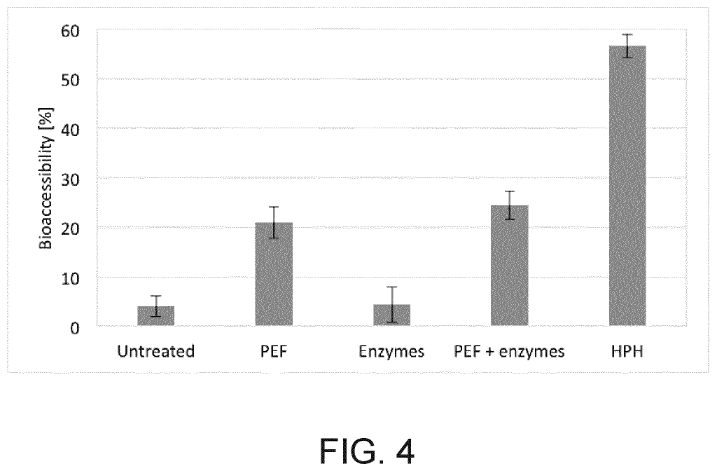

FIG. 4 shows that HPH led to the highest lipid bioaccessibility. Interestingly, the incubation after this treatment did not have any effect on the lipid bioaccessibility: being HPH without incubation 55.4±2.4% and HPH with 24 h incubation 56.5±2.2%. The mechanical disruption of algae cells by HPH is severe and fast and leads to an almost instantaneous diffusion of the intracellular compounds into the aqueous phase. The lipid fraction was already made accessible without the need for additional incubation.

PEF treatment showed an increase in lipid bioaccessibility by 17%, whereas enzymatic treatment did not have any relevant effect on lipid bioaccessibility (4.3%±3.6%) compared to the control (4.0% t 2.1%).

When the enzymes were added to the biomass in combination with a PEF treatment, no clear effect was shown (24.4%±2.8%), compared to PEF-treated biomass (20.9%±3.2%). In this experiment, the enzymes were added after the PEF treatment for practical reasons, and because no difference was reported when enzymes were added before the PEF treatment (data not shown).

### Example 7

### Particle Size

Cell integrity was determined in triplicate (n=3) by measuring the particle size of treated fresh microalgae cells with an LS 13 320 laser diffraction particle size analyser (Beckman Coulter, Brea, Canada). The results were displayed as mean diameter of the volume based droplet size distribution (d₄₃).

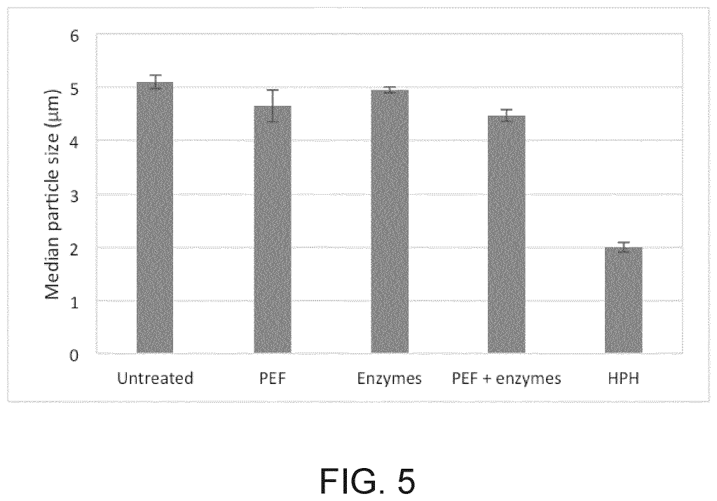

FIG. 5 shows the mean particle diameters of C. vulgaris cells untreated (control), PEF treated, enzymatically treated (chitinase+rhamnohydrolase+galactanase), PEF+enzymatically treated, and treated with HPH at 100 MPa. PEF (4.7±0.3 μm) and enzymatic (5.0±0.1 μm) treatments only led to a minor decrease in particle diameter compared to the control (5.1±0.1 μm). PEF treatment is preserving the cellular integrity and results in visually intact microalgae cells. Cell intactness would be maintained even if an autolytic process occurred after PEF treatment, as shown in yeast where autolysis induced cell-wall degradation without full cell break-down. In contrast, HPH treatment at 100 MPa reduced the average particle diameter to 2±0.1 μm, causing major disintegration of microalgae cells, being a mean particle size of 2.22 μm in C. vulgaris after 5 passes at 150 MPa. The combination of PEF and enzymes decreased the particle diameter to 4.5±0.1 μm. Hence, it seems that PEF treatment facilitated the action of enzymes on the cell wall polysaccharides, resulting in a slightly greater cell wall disintegration than with enzymes and PEF alone.

FIG. 5 shows the mean diameter of the volume based droplet size distribution (d43) of C. vulgaris cells untreated (control), PEF treated, enzymatically treated (chitinase+rhamnohydrolase+galactanase), PEF+enzymatically treated, and treated with HPH at 100 MPa. Error bars represent the standard deviation of triplicates (n=3).

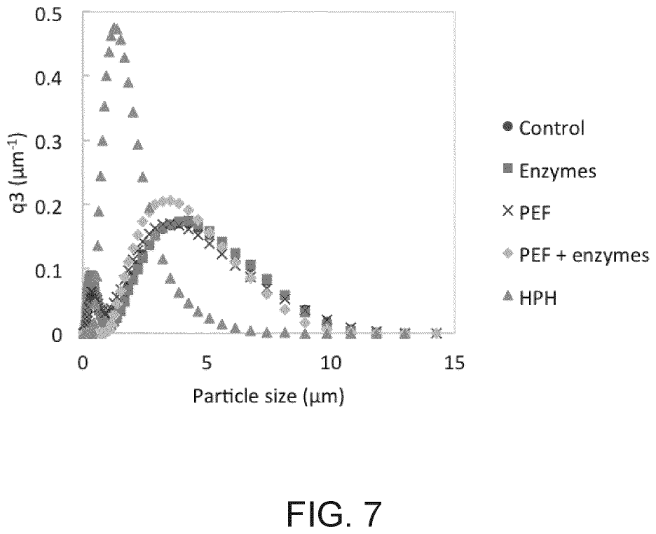

FIG. 7 shows the particle size distribution expressed as volume density (q3, μm⁻¹) of C. vulgaris cells that were untreated (control, ●), enzymatically treated (chitinase+rhamnohydrolase+galactanase, ▪), treated with PEF (×), PEF+enzymes (♦), and treated with high-pressure homogenization (HPH, A).

### Example 8

### Microbial Growth

The microbial growth (total viable aerobic count) of PEF treated biomass (incubated at 4° C., 48 h) was measured in biological triplicates. The biomass was diluted (10⁻¹, 10⁻², 10⁻³) with Difco™ maximum recovery diluent in duplicates and 25 μL were plated on universal growth medium and subsequently incubated under psychrotrophic (4° C.) and mesophilic (30° C.) conditions. Colony forming units (CFU) were counted after 24 h and 48 h whereby plates with CFU counts between 20-200 were considered as relevant.

In Table 1, the microbial growth is displayed in colony forming units per mL of biomass (CFU mL⁻¹). The growth was far below the minimum limit value (100 CFU g⁻¹) which counts as satisfactory according to of the swiss federal department of home affairs. The referred guideline is for Escherichia coli, which is an exemplary pathogenic mesophilic bacteria and thus suits well for comparison.

Table 1 shows the microbial growth in C. vulgaris suspension PEF treated and incubated (48 h, 4° C.) that was plated and incubated at 4 and 30° C. for up to 48 h. Data are the mean values of biological triplicates and technical duplicates (n=6).

| Plate incubation time | Plate incubation temperature | Microbial growth |
|---|---|---|
| h | ° C. | CFU mL⁻¹ |
| 24 | 4 | 0 |
| | 30 | 0 |
| 48 | 4 | 0 |
| | 30 | <1 |

The absence of microbial growth in the treated and incubated algae excluded the possibility of enzymatic activity coming from external bacteria that could be present in the suspension. This result could further support our hypothesis of enzymatic cell-wall degrading activity intrinsically present in C. vulgaris cells and triggered by PEF. Moreover, these data suggested the relevance of the here presented process (PEF followed by incubation at 4° C.) from a food safety perspective.

### Example 9

### Lipid Oxidative Stability

Lipid oxidation was studied by measuring secondary lipid oxidation products. In brief, treated biomass was freeze-dried and kept in amber vials (60 mg), and stored at 40° C. for 0, 2, 4, 6, 8, and 12 weeks. The secondary oxidation was assessed in triplicate (n=3). The biomass (60 mg) was dispersed in 1.5 mL of chloroform/methanol (1/2, v v⁻¹). The samples were mixed (2500 rpm, 10 min) in a multi-tube vortexer (DVX-2500, VWR, Switzerland). The sample supernatant (100 μL) was combined with 100 μL of 7-(diethylamino)-2-oxochromene-3-carbohydrazide (CHH) and 5 μL of internal standard (ISTD, hexanal-d₁₂, 10 μg mL-1 in acetonitrile). After an incubation for 1.5 h at 1400 rpm and 37° C. using a thermomixer (Comfort, Eppendorf, Schönenbuch, Switzerland), the sample was diluted with acetonitrile (100 μL) and centrifuged (2500×g, 20° C.). The supernatant was injected in an ultra-performance liquid chromatography (UPLC) (Dionex UltiMate 3000)-QExactive Plus (Thermo Scientific, Basel, Switzerland) system. (Z)-3-hexenal and hexanal were chosen as indicators for lipid oxidation, as volatiles coming from ω-3 and ω-6 fatty acid degradation pathways, respectively. The response factors were expressed as the ratio of the area of the volatiles to the area of the internal standard hexanal-d₁₂.

In addition, the stored dried samples were coded, randomized, and evaluated by sniffing by a panel of four people, who evaluated the odour by ranking the samples and describing the perceived flavour notes.

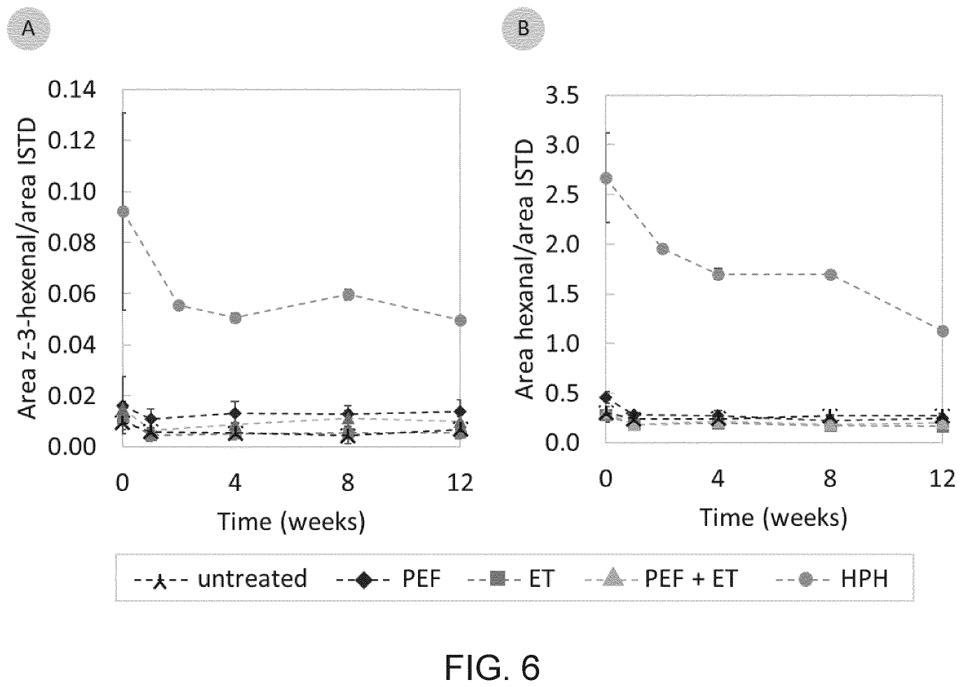

The production of volatiles was examined in the untreated biomass (control), PEF treated, enzymatically treated (chitinase+rhamnohydrolase+galactanase), PEF+enzymatically treated, and HPH treated. Hexanal and (Z)-3-hexenal values are shown in FIG. 6, as targeted degradation products of the most abundant fatty acids in C. vulgaris, named linoleic acid (C18:2,n6) and α-linolenic acid (C18:3,n3), respectively.

FIG. 6 shows the evolution of secondary oxidation products—A) (Z)-3-hexenal and B) hexanal—during 12 weeks of storage at 40° C. for C. vulgaris biomass untreated (control) ( ), PEF treated (♦), enzymatically treated (ET, chitinase+rhamnohydrolase+galactanase) (▪), PEF+enzymatically treated (▴), and treated HPH at 100 MPa (●). The signal is expressed as area of the targeted compound divided by the area of the internal standard (ISTD) hexanal-d12. Error bars represent the standard deviation of triplicates (n=3).

The signal of (Z)-3-hexenal and hexanal in untreated, PEF treated, ET treated and PEF+ET treated biomasses did not show an increase over 12 weeks of storage, indicating a remarkable oxidative stability. These results indicate that both PEF and ET are mild processes that preserve the quality of the lipid fraction. The signal intensity in HPH-treated biomass of hexanal and (Z)-3-hexenal was, respectively, 4.1 and 7.3 folds higher than the control after 12 weeks, indicating the harsh effect that HPH had on the biomass oxidative stability. Both hexane and (Z)-3-hexenal signals decreased from week 0 to week 4, which may be an indication of extensive oxidation already occurring through the disruption treatment, prior to the storage. Some of the produced aldehydes could have turned into alcohols and organic acids, which were not detected with the current analysis.

The sensory outcomes agreed with the analytical data. HPH treated biomass presented a rancid flavour already at time 0, whereas the untreated as well as enzymatic and PEF treated biomasses showed a subtle smell over the entire storage period. This demonstrates the potential of PEF treatment. Employing this technique can improve the lipid bioaccessibility in C. vulgaris biomass, while preserving its oxidative stability.

1. A method of making a product for human consumption which comprises partially lysed microalgae, said method comprising
Preparing a suspension of microalgae, wherein the microalgae belong to a phyla selected from the group consisting of Chlorophyta, Ochrophyta, and Heterokonta;
Applying a pulsed electric field to the suspension of microalgae, wherein said pulsed electric field has a specific energy input between 25 to 150 kJ per kg suspension (kg ₛᵤₛ⁻¹);
Forming a biomass of partially lysed microalgae; and
Adding the biomass of partially lysed microalgae as an ingredient of a product for human consumption.

2. The method according to claim 1, wherein the microalgae belong to the species Chlorella or Auxenochlorella.

3. The method according to claim 1, wherein the microalgae is Chlorella vulgaris.

4. The method according to claim 1, wherein the enzyme is endo-1,4-β-galactanase, rhamnogalacturonan rhamnohydrolase and chitinase.

5. The method according to claim 1, wherein the temperature of the suspension of microalgae before applying the pulsed electric field is between 2 to 30° C.

6. The method according to claim 1, wherein the pulsed electric field has a specific energy input between 25 to 100 kJ kg ₛᵤₛ⁻¹.

7. The method according to claim 1, wherein the pulsed electric field has an electric field strength between 10 kV cm cm⁻¹ to 45 kV cm cm⁻¹.

8. The method according to claim 1, wherein the pulsed electric field has a pulse length between 5 μs to 25 μs.

9. The method according to claim 1, wherein the pulsed electric field has a pulse number between 5 and 30 applied.

10. The method according to claim 1, wherein the pulsed electric field comprises bipolar square wave electric pulses, unipolar electric pulses, or exponential decay electric pulses.

11. The method according to claim 1, wherein the microalgae are collected and re-suspended in buffer after applying the pulsed electric field, wherein the buffer has a temperature between 4° C. to 37° C., and the microalgae are incubated in the buffer between 6 to 72 hours.

12. The method according to claim 11, wherein the buffer is a phosphate buffer.

13. The method according to claim 1, wherein the microalgae have a mean particle size between 3 μm to 6 μm after applying the pulsed electric field and/or enzyme treatment.

14. A method for improving lipid bioaccessibility of microalgae for human consumption, said method comprising
a. Preparing a suspension of microalgae, wherein the microalgae belong to a phyla selected from the group consisting of Chlorophyta, Ochrophyta, and Heterokonta;
b. Applying a pulsed electric field to the suspension of microalgae, wherein said pulsed electric field has a specific energy input between 25 to 150 kJ per kg suspension (kg ₛᵤₛ⁻¹);
the suspension of microalgae is incubated at between 4° C. to 37° C. for between 6 h to 48 h after step b).

15. A method for preserving lipid oxidative stability in microalgae for human consumption, said method comprising
a. Preparing a suspension of microalgae, wherein the microalgae belong to a phyla selected from the group consisting of Chlorophyta, Ochrophyta, and Heterokonta;
b. Applying a pulsed electric field to the suspension of microalgae, wherein said pulsed electric field has a specific energy input between 25 to 150 kJ per kg suspension (kg ₛᵤₛ⁻¹); and
c. adding enzyme to the suspension of microalgae.

16-17. (canceled)
